(19) United States
(12) Patent Application Publication
(10) Pub. No.: US 2013/0152865 A1
(43) Pub. Date: Jun. 20, 2013

(54) NUCLEUS COATED WITH A FILM-FORMING COATING HAVING ANTIBACTERIAL AND CICATRIZING PROPERTIES, AND METHOD FOR OBTAINING SAME

(71) Applicants: Jean Guezennec, Plouzane (FR); Yannick Gueguen, Saint Maur-des-Fosses (FR); Evelyne Bachere, Sainte Croix Vallee Francaise (FR); Achraf Kouzayha, Kalamoun (LB); Christelle Simon-Colin, Guilers (FR)

(72) Inventors: Jean Guezennec, Plouzane (FR); Yannick Gueguen, Saint Maur-des-Fosses (FR); Evelyne Bachere, Sainte Croix Vallee Francaise (FR); Achraf Kouzayha, Kalamoun (LB); Christelle Simon-Colin, Guilers (FR)

(73) Assignee: DIRECTION DES RESSOURCES MARINES, Papeete (FR)

(21) Appl. No.: 13/819,367

(22) PCT Filed: Aug. 31, 2011
(86) PCT No.: PCT/FR11/51994

§ 371 Date: Feb. 27, 2013

(30) Foreign Application Priority Data
Aug. 31, 2010 (FR) 1056889

(51) Int. Cl. A01K 61/00; A01K 67/033; A61K 31/7088

(52) U.S. Cl. CPC A01K 61/002; A61K 31/7088; A01K 67/0334
USPC 119/244; 424/400; 514/44 R; 800/13; 427/212

(57) ABSTRACT

A nucleus coated with a film includes one or more exopolysaccharides (EPS) and optionally one or more bioactive molecules such as bactericidal or bacteriostatic agents, cicatrizing agents and/or anti-inflammatory agents. A method for obtaining the nucleus, in order to inhibit graft rejection in pearl oysters is also described.

### FIELD OF THE INVENTION

The present invention relates to the field of nucleus coating in the context of pearl culture (production of pearls by pearl oysters). The present invention therefore relates to a nucleus coated with a film having healing and antibacterial properties, and enabling a reduction in mortality due to the insertion of said nucleus into the recipient pearl oyster and the phenomenon of nucleus rejection.

### PRIOR ART OF THE INVENTION

Pearl culture is a human activity consisting of culturing, in a natural environment, Pinctada sp pearl oysters in order to produce cultured pearls. The first step involves collecting and farming pearl oysters, which will be used either as donor oysters or recipient oysters. The graft consists of a surgical operation in which the graft, a portion of the epithelium of the mantle of the donor oyster (about 4 mm²) is inserted into the pearl pouch of the recipient oyster, in combination with a nacre bead, the nucleus. Once inserted into the recipient oyster, the epithelial lining of the graft multiples and covers the pearl pouch so as to produce the pearl sac that surrounds the nucleus. Said pearl sac deposits layers of nacre around the nucleus, resulting in the production of the pearl (Montagnani et al., 2009).

Mortality and nucleus rejections occur for around 45 days after the graft, at variable rates. These phenomena, which affect 40 to 50% of oysters within three weeks following the operation, appear to result from infectious pathologies or from an inappropriate grafting practice. The development of an inflammatory reaction after insertion of the nucleus and contamination by pathogenic bacteria, combined with the absence of rapid healing of the tissue incised during the graft are probably the main causes of nucleus rejection (Cochennec et al., 2010).

There is therefore a need for processes resulting in lower rates of failure of grafting operations, in particular by reducing mortality and nucleus rejection.

Patent application JP05219856 describes the insertion, in the recipient oyster, during the grafting step and in combination with the nucleus or by means of the nucleus, of a solid material containing, at its surface, a polymer associated with an antibiotic.

Japanese patent applications JP02308869 and JP63215609 claim the use of a nucleus covered with a polymer in order to improve in particular the quality of the pearls obtained, by increasing the homogeneity of the surface of the nucleus, and for reducing rejection and mortality phenomena, by avoiding, for example, colonization of the nucleus by various parasites.

Japanese patent application JP02174621 describes the use of a natural polymer for coating the nucleus, associated with an antifungal agent, in order to limit contaminations during grafting.

Patent U.S. Pat. No. 6,514,614 describes the coating of the nucleus with a water-soluble polymer, associated with a substance having antibacterial activity, said polymer being partially dissolved by seawater (the dissolution rate being greater than 25%) so as to enable an effective reduction in friction and resistance to insertion of said nucleus.

Finally, Japanese patent application JP03183424 describes a system for coating the nucleus with a water-soluble polymer (or by any other compound enabling delayed administration), enabling the administration, at a controlled rate, of an antibiotic associated with said polymer.

However, the publications cited above do not solve the problem of healing of the incision formed during the graft. Moreover, the use of antibiotics presents an environmental problem, due to their incomplete biodegradability, and a public health problem because of the development of bacterial resistance.

Surprisingly, the applicant has noted that the coating of the nucleus with an EPS (exopolysaccharide) film enables the failure rate of graft operations to be reduced. Without willing to be bound to any theory, the coating of the nucleus by an EPS film would make it possible to accelerate the healing of the recipient oyster. Moreover, the association of said EPS film with bactericidal or bacteriostatic agents, such as, for example, antimicrobial peptides (AMP), enables the occurrence of microbial contaminations to be reduced. The presence of EPS alone or in combination with a bactericidal or bacteriostatic agent thus enables failures of the grafting step to be reduced, and in particular mortality of the recipient oysters and nucleus rejection by said oysters to be reduced.

### SUMMARY

The invention relates to a nucleus coated with a film comprising one or more exopolysaccharide(s) (EPS).

According to an embodiment of the invention, the EPS are produced by gram-positive or gram-negative bacteria, archaea or algae.

According to an embodiment of the invention, the film further comprises one or more bioactive molecule(s).

According to an embodiment of the invention, said bioactive molecules are chosen among one or more bactericidal or bacteriostatic agent(s), one or more healing agent(s) and/or one or more anti-inflammatory agent(s).

According to an embodiment of the invention, the EPS are chosen among HE 800, EPS 721, MO245, GG1, HYD 657, HYD 1644, HYD 1545, GY 785, MS 907, ST 716, HYD 721, GY 772, HYD 750, GY 768, GY 788, BI746, GY 786, GY 685, GY 686, ST 719, HYD 1574, HYD 1579, HYD 1582, HYD 1584, ST 708, ST 722, ST 342, ST 349, HYD 1625, and HYD 1666, and preferably MO 245, HE 800, GG1, HYD 721 and ST 716.

According to an embodiment of the invention, the bactericidal or bacteriostatic agent(s) are chosen from the antimicrobial peptides (AMP).

According to an embodiment of the invention, said AMP is chosen among tachyplesin or the oyster defensins, Cg-Defs.

The invention also relates to a process for obtaining the nucleus as described above, said process comprising a step of coating the nucleus with EPS by immersion in a solution containing 0.1 to 10% in weight of EPS to the volume of the solution.

According to an embodiment of the invention, the process comprises:
a first step as described above, and
a second step of associating one or more bactericidal or bacteriostatic agent(s) with the EPS film previously formed, by immersion in a solution comprising 1 to 10 MIC of said bactericidal or bacteriostatic agent(s).

According to another embodiment, the process for obtaining the nucleus coated with a film comprising one or more EPS in combination with one or more bactericidal or bacteriostatic agent(s) comprises a step of coating the nucleus with the EPS and the bactericidal or bacteriostatic agents, by immersion in a solution comprising 0.05 to 10% in weight of EPS to the volume of the solution and 1 to 10 MIC of said bactericidal or bacteriostatic agent(s).

The invention also relates to a pearl oyster comprising a nucleus as described above.

The invention also relates to the use of the nucleus as described above or the process as described above for reducing the failure of the graft of a recipient oyster with a nucleus, said failure consisting in mortality or rejection of the nucleus by the recipient pearl oyster.

The invention also relates to a process for grafting a recipient pearl oyster comprising the insertion of a graft into the pearl pouch of the recipient pearl oyster, which graft corresponds to the epithelium of the mantle of the donor oyster, in combination with a nucleus as described above, or with a nucleus obtained by a process as described above.

The invention also relates to a process for producing a cultured pearl, comprising the grafting process as described above.

The invention also relates to a process for obtaining pearls, comprising the grafting of a nucleus according to the invention, or a nucleus obtained by a process according to the invention, in combination with a portion of the epithelium of the mantle of the donor oyster in the pearl pouch of the recipient oyster.

The invention also relates to a pearl obtained by the process for obtaining pearls as described above, or comprising the nucleus according to the invention or a nucleus obtained by a process according to the invention, or comprising a film comprising one or more exopolysaccharide(s) (EPS) produced by gram-positive or gram-negative bacteria, archaea or algae under one or more nacre thicknesses.

The present invention also relates to a process for improving the quality of the pearl and/or for reducing the failure of the graft of a recipient oyster with a nucleus, said failure corresponding to mortality or rejection of the nucleus by the recipient pearl oyster, and said process comprising the grafting of the recipient oyster with a nucleus according to the invention or with a nucleus obtained by the process for obtaining a nucleus according to the invention.

The present invention also relates to the use of the nucleus according to the invention or of the nucleus obtained by the process for obtaining a nucleus according to the invention for reducing the failure of the graft of a recipient oyster with a nucleus, said failure consisting in mortality or rejection of the nucleus by the recipient pearl oyster and/or for improving the quality of the pearl obtained.

### DEFINITIONS

In this invention, the following terms have the following meanings:
“Nucleus”: element, generally spherical, formed by a natural compound (for example, the nacre of the mussel of Mississippi, Amblema plicata) or a synthetic compound (for example, Bironite) inserted into the recipient oyster during the grafting step, and serving as a support for the deposition of nacre by the recipient pearl oyster. Said nuclei may be coated with specific substances so as to improve the quality of the pearl obtained.
“Pearl culture”: human activity with the objective of producing cultured pearls by pearl oysters, generally belonging to the genus Pinctada sp.
“Bactericidal agent”: substance having a bactericidal action, i.e. causing bacteria death.
“Bacteriostatic agent”: substance having a bacteriostatic action, i e inhibiting development, growth and/or division of bacteria.
“MIC”: minimum inhibitory concentration. This corresponds to the minimum concentration at which an agent inhibits the visible growth of a microorganism after incubation overnight. The methods for determining the MIC of an agent are well known in the state of art.
“Healing”: describes an agent capable of promoting the recovery or healing of a wound, more specifically an open wound, such as an incision.
“About”: placed before a numeric element, means more or less 10% of this numeric element.
“Nacre”: biomineralized structure formed by aragonite crystals (forming about 90% of the nacre) and conchiolin. Aragonite is a chemical compound of formula CaCO₃+traces of Sr; Pb; Zn.

### DETAILED DESCRIPTION

In pearl culture, the production of the pearl results from the deposition of nacre on a nucleus inserted into the recipient oyster during the grafting step. The step of grafting the nucleus generally has a high failure rate, due to poor healing of the recipient oyster at the incision created during the graft or bacterial contaminations. Thus, the nuclei used in pearl culture are increasingly covered with a coating enabling the failure rate of the grafting step to be reduced.

This invention therefore relates to a nucleus covered or coated with a film comprising one or more exopolysaccharides (EPS).

The EPS film would have healing properties, enabling the healing of wounds induced by the grafting step to be accelerated, and therefore the mortality of the recipient oyster leading to failure of the graft to be reduced.

It has been demonstrated that some bacteria have the capacity to synthesize, under controlled conditions, exopolymers including exopolysaccharides (EPS), in response to nutritional imbalance conditions. Exopolysaccharides can be defined as macromolecules formed by linking similar carbohydrates (commonly called sugars or simple sugars). These exopolysaccharides can be produced on an industrial scale, and have, among other properties, adhesive (associated with the function of these molecules in nature) and film-forming properties.

EPS can be produced by numerous microorganisms such as gram-positive or gram-negative bacteria, archaea, fungi and some algae. Advantageously, EPS are produced by gram-positive or gram-negative bacteria, archaea or algae.

EPS can be extracted from microorganism cultures by well known physical or chemical extraction methods such as, for example, sonication, centrifugation, alkaline treatment, ethanol extraction, enzymatic extraction . . .

According to an embodiment of the invention, the EPS can be chosen among those produced by marine organisms such as Bacillus, Halomonas, Planococcus, Enterobacter, Alteromonas, Psuedoalteromonas, Rhodococcus, Zoogloea, Cyanobacteria, Vibrio, as described in Satpute et al. (Biotechnology Advances 2010, 28; 436-450). If the taxonomy had to be modified, a person skilled in the art could adapt the taxonomy modifications in order to deduce therefrom the EPS capable of being used in the invention.

According to an embodiment of the invention, the EPS can be obtained by fermentation of bacteria from deep-sea hydrothermal ecosystems. More specifically, these EPS are those synthesized under controlled conditions (nutritional imbalance generated by a high carbon/nitrogen ratio due to a carbohydrate-enriched nutritional environment) during fermentation of bacteria from deep-sea hydrothermal ecosystems (see, for example, Guezennec, J. (2002). Deep-sea hydrothermal vents: A new source of innovative bacterial exopolysaccharides of biotechnological interest? Journal of Industrial Microbiology & Biotechnology 29: 204-208).

According to an embodiment, the EPS are chosen from the group comprising uncharged (or neutral) EPS, such as, for example, GG1.

According to an embodiment, the EPS are chosen from the group comprising charged EPS, such as, for example, HE800 and MO245.

According to an embodiment, the EPS can be chosen from HE 800, EPS 721, MO245, GG1, HYD 657, HYD 1644, HYD 1545, GY 785, MS 907, ST 716, HYD 721, GY 772, HYD 750, GY 768, GY 788, BI746, GY 786, GY 685, GY 686, ST 719, HYD 1574, HYD 1579, HYD 1582, HYD 1584, ST 708, ST 722, ST 342, ST 349, HYD 1625, and HYD 1666, preferably MO 245, HE 800, GG1, HYD 721 and ST 716.

According to an embodiment, the EPS are native. According to another embodiment, the EPS can be chemically or physically modified (such as, for example, by adding sulfate, acetate, lactate, succinate or pyruvate groups).

According to an embodiment of the invention, the EPS film is continuous around the nucleus.

The invention also relates to a nucleus coated with a film comprising one or more EPS and one or more bioactive molecule(s).

According to an embodiment, these bioactive molecules are bactericidal or bacteriostatic agents. In pearl culture, the addition of a bactericidal or bacteriostatic agent on the nucleus enables the occurrence of bacterial contamination in the recipient oyster, capable of resulting in rejection of the nucleus or death of said oyster, to be limited.

According to another embodiment, these bioactive molecules are healing or anti-inflammatory agents such as collagen, fibrinogen, laminin or growth factors.

According to an embodiment of the invention, the bactericidal or bacteriostatic agents are chosen among the chemical antibiotics such as, for example, tetracycline, kanamycin, sulfamonomethoxine, and ampicillin.

According to another embodiment of the invention, the bactericidal or bacteriostatic agents are chosen among the antimicrobial peptides (AMP).

The antimicrobial peptides (AMP) are effector molecules of innate immunity, preserved over evolution and widespread throughout the entire living kingdom. A wide variety of AMPs has been identified in recent years, showing great diversity in terms of structure, size and modes of action. AMPs are generally characterized by a high representation of cationic and hydrophobic amino acids. These molecules usually have an amphiphilic character essential for their interaction with the bacterial membranes (Bulet et al. 2004). The AMPs kill the microorganisms, by permeabilizing their membrane by a detergent-type effect or by forming pores, or by inhibiting the synthesis of peptidoglycan constituting the bacterial wall, or by inhibiting the bacterial metabolic pathways (Brodgen et al., 2005).

With respect to the chemical antibiotics generally used, AMPs have the advantage of being entirely biodegradable. They appear to be good candidates for substitution for conventional chemical antibiotics, due to their biological properties. Indeed, they have a broad spectrum of antimicrobial activity, low specificity, different modes of action and are harmless to the environment.

AMPs can be produced by chemical synthesis or by expression in a bacterial or yeast recombinant system (cloning, expression, purification).

According to an embodiment of the invention, AMPs may belong to the family of linear AMP with an alpha helix, the AMP family having an overrepresentation of one or more amino acids, the hairpin AMP family (beta hairpin) with 1 or 2 disulfide bonds, or the cyclic AMP family with a beta sheet and an alpha helix with 3 or more disulfide bonds (Bulet et al., Immunological Reviews, 2004, 198: 169-184; Brogden, Nature Review Microbiology, 2005, 3: 238-250).

Examples of linear AMPs with an alpha helix include, but are not limited to, cecropin, stomoxyn, ponericin, spinigerin, oxyopinin, cupiennin, clavanin, styelin, pardaxin, misgurin, pleurocidin, parasin, oncorhyncin, moronecidin, magainin, temporin, cathelicidin and indolicidin.

Examples of AMPs enriched with one or more amino acid, proline, arginine, glycine or tryptophan, include, but are not limited to, bactenecins, PR-39, abaecins, apidaecins, drosocin, pyrrhocoricins, Cg-Prp, prophenin and indolicin.

Examples of hairpin AMPs containing 2 to 4 cysteines include, but are not limited to, tachyplesin, protegrin, thanatin, androctonin, gomesin, polyphemusin, hepcidin, brevinin, esculentin, tigerinin and bactenecin.

Examples of cyclic AMPs containing 6 or more cysteine residues or with an open cycle include, but are not limited to, defensins (vertebrate, invertebrate or plant), termicin, heliomicin, drosomycin, ASABF, pBD, penaeidins, ALF and big-defensins. Examples of invertebrate defensins are the Cg-Defs oyster defensins or the MGD mussel defensins.

According to an embodiment of the invention, the AMP is chosen among tachyplesin and the Cg-Defs oyster defensins.

According to an embodiment of the invention, the AMPs are synthesized by chemical synthesis. According to another embodiment of the invention, the AMPs are synthesized by biological synthesis in a bacterial or fungal recombinant system and, preferably, in a yeast system.

Mortality or nucleus rejection resulting from the graft of the recipient oyster generally occurs in the 45 days after the operation, and usually in the first three weeks after the operation.

According to an embodiment of the invention, the AMPs are stably associated with said EPS film.

According to an embodiment of the invention, the film comprising EPS and optionally bacteriostatic or bactericidal agents resists washing with seawater and is stable for more than three weeks, preferably more than one month at temperatures ranging from 4 to 30° C. Thus, the film comprising EPS and optionally bacteriostatic or bactericidal agents is not dissolved during contact with seawater (Example 1), for at least three weeks.

According to an embodiment of the invention, the film comprising EPS and optionally bacteriostatic or bactericidal agents preserves a bacterial growth inhibiting activity for more than three weeks, preferably more than one month, at a temperature of 37° C.

According to an embodiment of the invention, the association between the EPS film and the bacteriostatic or bactericidal agent(s) such as AMPs results from an adsorption phenomenon or a chemical reaction between the EPS and said agents.

This invention also relates to a process for coating the pearl culture nucleus with a film comprising one or more exopolysaccharides of bacterial origin.

According to an embodiment of the invention, said process comprises a first step of immersing the nucleus in an aqueous EPS solution, preferably a solution of EPS in reverse osmosis water. According to a preferred embodiment of the invention, said aqueous EPS solution has an EPS concentration from 0.1 to 10% weight/volume, more preferably from 0.5 to 5%, even more preferably of 1% by weight of EPS per volume of the aqueous solution.

According to an embodiment of the invention, said first step of immersing the nucleus in a solution comprising one or more EPS is performed at a constant temperature, preferably at ambient temperature (i.e. 15 to 25° C.), more preferably about 20° C. According to this embodiment of the invention, said first step of immersing the nucleus in a solution comprising one or more EPS is performed preferably for 1 to 60 minutes, more preferably for 5 to 20 minutes, even more preferably for 10 minutes.

According to another embodiment of the invention, said first step of immersing the nucleus in a solution comprising one or more EPS is performed at a constant temperature, preferably from 1 to 10° C., more preferably about 4° C. According to this embodiment of the invention, said first step of immersing the nucleus in a solution containing one or more EPS is performed preferably for 1 to 3 hours, more preferably for about 2 hours.

According to an embodiment of the invention, the coated nucleus is then dried under vacuum.

When the film coating the nucleus further comprises one or more bactericidal or bacteriostatic agent(s), the first step described above is followed by a second step comprising the immersion of the nucleus in a solution comprising one or more bactericidal or bacteriostatic agent(s) at a concentration from 1 to 10 MIC, preferably of 10 MIC.

According to the invention, the bactericidal or bacteriostatic agent is in solution in a biologically acceptable polar solvent such as water (for example, reverse osmosis water), ethanol, trifluoroethanol (CF₃CH₂OH, TFE) or a mixture thereof such as, for example, water/TFE, preferably trifluoroethanol (CF₃CH₂OH) or reverse osmosis water.

According to a first embodiment of the invention, the second step of immersing the nucleus is performed at a constant temperature, preferably from 1 to 10° C., more preferably about 4° C. According to one embodiment of the invention, said second step of immersing the nucleus is performed preferably for 24 to 120 hours, more preferably for 48 to 96 hours, even more preferably for 72 hours. According to another embodiment, said second step of immersing the nucleus is performed for 12 to 48 hours, preferably 12 to 24 hours and more preferably 24 hours.

According to a second embodiment of the invention, the second step of immersing the nucleus is performed at a constant temperature, preferably at ambient temperature (from 20 to 30° C.) more preferably about 25° C. According to an embodiment of the invention, said second step of immersing the nucleus is performed preferably for 30 minutes to 3 hours, preferably for 1 to 2 hours, and even more preferably for about 1 hour 30 minutes.

According to an embodiment of the invention, the coated nucleus is then dried under vacuum.

According to an embodiment of the invention, said process optionally comprises a step of rinsing the nucleus between the first immersion step and the second immersion step. According to this embodiment, the nucleus is rinsed with a volume of 10 to 1000 mL of distilled water, preferably 100 to 300 mL of distilled water, more preferably about 200 mL of distilled water.

The invention also relates to a process for obtaining a nucleus coated with a film comprising one or more EPS and one or more bacteriostatic or bactericidal agent(s), said process comprising a single step of immersing the nucleus in a solution comprising EPS and bacteriostatic or bactericidal agents.

According to an embodiment, the EPS are present in the solution at a concentration from 0.05 to 10% in weight to the total volume of the solution, preferably from 0.1 to 5%, more preferably at a concentration of 1%.

According to an embodiment, the bactericidal or bacteriostatic agents are present in the solution at a concentration from 1 to 10 MIC.

According to an embodiment, this immersion step is performed at a constant temperature, from 1 to 10° C., preferably 4° C., and for 12 to 128 hours, preferably 48 to 96 hours, and more preferably for 72 hours.

According to an embodiment of the invention, the coated nucleus according to the invention, or obtained according to a process of the invention, is preserved at 4° C. According to another embodiment of the invention, the coated nucleus according to the invention, or obtained according to a process of the invention, is preserved at ambient temperature.

According to an embodiment of the invention, the nucleus is of natural origin, more preferably said nucleus is formed by the nacre of a Mississippi mussel belonging to the Amblema sp. genus, preferably Amblema plicata. Examples of nuclei are those sold by Aming (Standard Aming) or Poe Import.

According to an embodiment of the invention, said nucleus has a diameter from 1 to 20 mm, preferably 2 to 15 mm, more preferably 2 to 4 mm, and even more preferably 2.1 to 3.5 mm.

According to an embodiment of the invention, said nucleus has a diameter from 2 to 2.5 BU, preferably about 2.4 BU. The BU is a unit of measurement, with 1 BU corresponding to 3.03 mm.

The invention also relates to an oyster comprising a nucleus as described above or obtained by a process as described above.

Preferably, the oyster belongs to the Pinctada sp. genus, more preferably to the Pinctada fucata, Pinctada maxima or Pinctada margaritifera species.

The invention also relates to a process for grafting a recipient pearl oyster comprising the insertion, into the pearl pouch of the recipient pearl oyster, of a graft, corresponding to the epithelium of the mantle of the donor oyster, in combination with a nucleus as described above, or obtained by a process as described above.

According to an embodiment of the invention, the grafting process comprises (i) the manual opening of the recipient pearl oyster, (ii) the creation of an incision in the tissues of the recipient pearl oyster in order to access the pearl pouch and enable, in a third step (iii), the insertion, into the pearl pouch of the recipient pearl oyster, of a graft, corresponding to a portion of the epithelium of the mantle of the donor oyster (about 4 mm²), in combination with a nucleus coated with an EPS film as described above.

The invention also relates to a process for producing a cultured pearl, comprising the grafting process as described above.

This invention also relates to a process for obtaining pearls comprising the use of the coated nucleus as described above, or obtained according to a coating process as described above.

According to an embodiment of the invention, the process for obtaining pearls comprises the grafting of a nucleus according to the invention in the pearl pouch of a recipient oyster, in combination with a portion of the epithelium of the mantle of a donor oyster.

According to an embodiment of the invention, said process for producing or obtaining pearls comprises a first step comprising the collection and farming of pearl oysters, preferably belonging to the genera and species cited above, in order to obtain donor pearl oysters and recipient pearl oysters.

According to an embodiment of the invention, the donor and recipient pearl oysters are then cleaned to remove any parasites.

According to an embodiment of the invention, said production process further comprises, after the graft, a step of culturing the recipient pearl oysters, preferably for a period of 10 to 24 months, preferably for 12 to 20 months, even more preferably for 16 to 18 months. During the culture period, the epithelial lining of the graft multiples and covers the pearl pouch, to produce a pearl sac surrounding the nucleus, and which will deposit layers of nacre around the nucleus, thus resulting in the production of a pearl.

The invention also relates to a pearl obtained by the process for obtaining pearls as described above.

The invention also relates to a pearl comprising a nucleus coated according to the invention, or a nucleus obtained by a process according to the invention.

This invention also relates to a pearl of which the nucleus is coated with an EPS film, preferably EPS produced by gram-positive or gram-negative bacteria, archaea or algae, optionally in association with one or more bioactive molecules.

This invention also relates to a pearl comprising an EPS film, preferably EPS produced by gram-positive or gram-negative bacteria, archaea or algae, optionally in association with one or more bioactive molecules under one or more nacre thicknesses.

According to an embodiment of the invention, the pearl has a size, preferably a diameter from 2 to 20 mm, preferably from 5 to 15 mm, even more preferably from 6.8 to 10 mm.

According to the invention, the presence of a film around the nucleus as described in the invention has a number of advantages:
Improvement in the homogeneity of the surface of the nucleus, and/or
Improvement in the quality of the pearl obtained (limitation in the appearance of surface defects) and/or
Acceleration of healing of the incision formed during the graft, and/or
Action on the cells of the graft and the pearl sac improving the development of the pearl, and/or
Inhibition of microbial contamination phenomena, and/or
Reduction in mortality of recipient oysters by infection of the incision.

This invention also relates to a process for improving the homogeneity of a nucleus, comprising the coating of said nucleus with a film as described in this invention.

This invention also relates to a process for improving the quality of the pearl obtained in the graft of a recipient oyster, comprising the use of a nucleus according to the invention. According to an embodiment of the invention, the use of the nucleus according to the invention during the grafting step limits the appearance of surface defects on the pearl.

This invention also relates to a process for reducing failure of the graft of a recipient oyster with a nucleus, said failure corresponding to mortality or rejection of the nucleus by the recipient pearl oyster, and said process comprising the grafting of the recipient oyster with a nucleus according to the invention or obtained by the process for obtaining a nucleus according to the invention.

This invention also relates to a process for inhibiting rejection of the nucleus during the graft, said process comprising the use of a coated nucleus according to the invention in the graft.

This invention further relates to a process for reducing mortality of recipient oysters resulting from the grafting step, comprising the use of a coated nucleus according to the invention in the graft. According to one embodiment of the invention, the mortality prevented by the process of the invention is due to an infection of the incision created during the graft.

This invention also relates to the use of the nucleus according to the invention or obtained by the process for obtaining a nucleus according to the invention for reducing the failure of the graft of a recipient oyster with a nucleus, said failure consisting of death or rejection of the nucleus by the recipient pearl oyster and/or for improving the quality of the pearl obtained.

This invention also relates to a process for accelerating healing of the incision produced during the graft, comprising the use of a coated nucleus according to the invention.

This invention also relates to a process for inhibiting microbial contamination phenomena occurring during the graft, said process comprising the use of a coated nucleus according to the invention in the graft.

In addition, the use of AMPs as bacteriostatic or bactericidal agents is better for the environment than the use of conventional chemical antibiotics, and for health, as the muscle of the pearl oyster may be consumed raw.

### BRIEF DESCRIPTION OF THE FIGURES

FIG. 1 is a set of photographs obtained by scanning microscopy, of uncoated nuclei (a) and coated nuclei (b, c and d). The marks correspond to intentional tears in order to confirm the presence of the film.

FIG. 2 is a set of photographs of Petri dishes after culture performed with bacterial solutions incubated with nuclei. (a) nucleus uncoated or coated with EPS alone. (b) nucleus coated with a combination of EPS/AMP without rinsing. (c) nucleus coated with a combination of EPS/AMP and rinsed with seawater or MilliQ water.

### EXAMPLES

This invention will be easier to understand and interpret in view of the following examples.

### Example 1: Presence of the EPS Film

Nuclei were coated with a film comprising the EPS MO245 and optionally tachyplesin. These nuclei were obtained by a two-step process:
1) immersion in a solution comprising 1% MO245 for 2 hours at 4° C.,
2) immersion in a TFE solution comprising 70 mg/l of tachyplesin (i.e. 10 MIC) for 72 hours at 4° C.

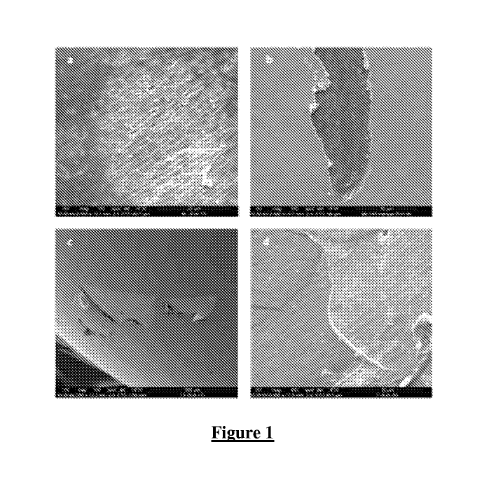

FIG. 1 obtained by scanning microscopy unambiguously shows the presence of a film. FIGS. b and c show the presence of the film after a tear and FIG. d shows that the film remains stable after washing with water and/or TFE.

### Example 2: Antimicrobial Activity

### Experiments:

Nuclei of standard diameter were used for the studies on the influence of a pretreatment of the surfaces with biopolymers obtained from bacterial fermentation.

The nuclei are coated with a film comprising the EPS MO245 alone or in combination with tachyplesin.

These nuclei are obtained by a single-step process: immersion of the nuclei in a solution comprising 0.1% MO245 and optionally 70 mg/l (i.e. 10 MIC) of tachyplesin for 48 hours at 4° C.

Certain nuclei were rinsed with 200 ml of seawater or MilliQ water before being dried. All of the nuclei were then dried under vacuum.

A conventional bacterial growth inhibition test was performed on these nuclei: the nuclei were placed in contact with a bacterial solution in the exponential phase of growth of the Vibrio strain (Vibrio Pmar02-149) isolated from Pinctada margaritifera in a graft in Tahiti for 18 hours at 30° C. The bacterial growth was then measured with a spectrophotometer at an optical density of 630 nm. The bacterial solution is then spread on a Petri dish (Zobell Agar medium) and the colonies developed are then counted.

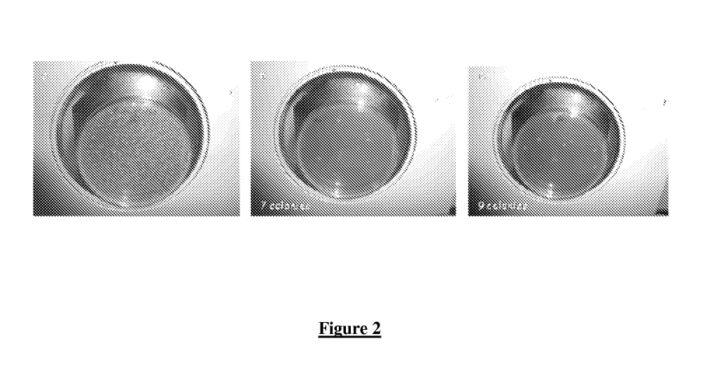

FIG. 2a shows that the presence of uncoated nuclei does not inhibit bacterial growth. Similar results were obtained with nuclei coated only with a film comprising EPS.

FIG. 2b shows that the presence of nuclei coated by a film comprising a combination of EPS+AMP inhibits bacterial growth.

FIG. 2c shows that the antibacterial activity of the nuclei coated with a film comprising a combination of EPS+AMP is preserved after washing of the nuclei.

In addition, the stability of the antimicrobial activity was studied over time and against the seawater treatment (essential element in the pearl cavity of the oyster). The results show that the antimicrobial activity of a nucleus coated with a film comprising a combination of EPS+AMP is stable for at least two weeks at ambient temperature (18 to 21° C.).

The same experiment was then performed on nuclei coated with the EPS MO245 or the EPS GG1 in combination with tachyplesin.

The antimicrobial activity of the nuclei is measured by the percentage of inhibition of bacterial growth after 18 hours of contact with respect to the control (bacterial culture without nuclei). The inhibition percentages measured are presented in the table below.

| Coating of the nucleus | % inhibition |
|---|---|
| EPS MO245 + tachyplesin | 97% |
| EPS GG1 + tachyplesin | 95% |

The nuclei coated with EPS in combination with AMP (tachyplesin) strongly inhibit bacterial growth (97 and 95% for the EPS MO245 and GG1, respectively).

### Example 3: Efficacy of the Nuclei of the Invention in Graft Experiments

### 1—Mortality

Uncoated nuclei, or nuclei coated with the EPS GG1, alone or in combination with an AMP (tachyplesin) were grafted in recipient oysters in combination with a portion of the epithelium of the mantle of a recipient oyster (graft). 67 donor oysters were used for this experiment. 8 graft experiments were performed per type of nucleus and per donor.

The mortality of the recipient oysters was evaluated 40 days after the graft. The mortality is evaluated as a percentage with respect to the oysters having received an uncoated nucleus. The results are presented in the table below:

| Condition | Mortality (%) |
|---|---|
| Uncoated nucleus | 100% |
| Nucleus coated with the EPS GG1 | 77.6% |
| Nucleus coated with the EPS GG1 | 76.1% |
| and tachyplesin | |

The presence of the EPS film or the EPS+AMP film on the nucleus inhibits the mortality of the oysters by almost 25%.

2—Graft rejection

Uncoated nuclei, or nuclei coated with the EPS MO245 and an AMP (tachyplesin) were used in graft experiments as described above.

The rejection of the nuclei 40 days post-graft was measured. The results are presented in the table below, and are expressed with respect to the oysters having received an uncoated nucleus.

| Condition | Rejection (%) |
|---|---|
| Uncoated nucleus | 100% |
| Nucleus coated with the EPS MO245 | 79.5% |
| and tachyplesin | |

The presence of a film according to the invention, comprising an EPS and an AMP, makes it possible to reduce the rejection of the nucleus by more than 20%.

1. A nucleus coated with a film comprising one or more exopolysaccharides (EPS) produced by gram-positive or gram-negative bacteria, archaea or algae.

2. The nucleus according to claim 1, characterized in that the film further comprises one or more bioactive molecule(s).

3. The nucleus according to claim 2, characterized in that the bioactive molecule(s) is (are) chosen among bactericidal or bacteriostatic agents, healing agents and/or anti-inflammatory agents.

4. The nucleus according to claim 1, characterized in that the EPS are chosen among HE 800, EPS 721, MO245, GG1, HYD 657, HYD 1644, HYD 1545, GY 785, MS 907, ST 716, HYD 721, GY 772, HYD 750, GY 768, GY 788, BI746, GY 786, GY 685, GY 686, ST 719, HYD 1574, HYD 1579, HYD 1582, HYD 1584, ST 708, ST 722, ST 342, ST 349, HYD 1625, and HYD 1666, preferably MO 245, HE 800, GG1, HYD 721 and ST 716.

5. The nucleus according to claim 3, characterized in that the bactericidal or bacteriostatic agent(s) is (are) chosen among the antimicrobial peptides (AMP), preferably from tachyplesin or the oyster defensins Cg-Defs.

6. A process for obtaining the nucleus according to claim 1, said process comprising a step of coating the nucleus with EPS by immersion in a solution containing 0.1 to 10% in weight of EPS to the total volume of the solution.

7. A process for obtaining the nucleus according to claim 2, characterized in that it comprises:
a first step of coating the nucleus with EPS by immersion in a solution containing 0.1 to 10% in weight of EPS to the total volume of the solution, and
a second step of associating one or more bactericidal or bacteriostatic agent(s) with the EPS film previously formed, by immersion in a solution comprising 1 to 10 MIC of said bactericidal or bacteriostatic agent(s).

8. A process for obtaining the nucleus according to claim 2, characterized in that it comprises a step of coating the nucleus with the EPS and the bactericidal or bacteriostatic agents, by immersion in a solution comprising 0.05 to 10% in weight of EPS to the volume of the solution and 1 to 10 MIC of said bactericidal or bacteriostatic agent(s).

9. Pearl oyster comprising a nucleus according to claim 1.

10. Process for grafting a recipient pearl oyster comprising the insertion of a graft into the pearl pouch of the recipient pearl oyster, which graft corresponds to the epithelium of the mantle of the donor oyster, in combination with a nucleus according to claim 1.

11. A process for producing a cultured pearl, comprising the process according to claim 10.

12. Pearl obtained according to claim 11, comprising a film comprising one or more exopolysaccharide(s) (EPS) produced by gram-positive or gram-negative bacteria, archaea or algae under one or more nacre thicknesses.

13. Process for improving the quality of the pearl and/or for reducing the failure of the graft of a recipient oyster with a nucleus, said failure corresponding to mortality or rejection of the nucleus by the recipient pearl oyster, and said process comprising the grafting of the recipient oyster with a nucleus according to claim 1.

14. (canceled)

15. The nucleus according to claim 2, characterized in that the EPS are chosen among HE 800, EPS 721, MO245, GG1, HYD 657, HYD 1644, HYD 1545, GY 785, MS 907, ST 716, HYD 721, GY 772, HYD 750, GY 768, GY 788, BI746, GY 786, GY 685, GY 686, ST 719, HYD 1574, HYD 1579, HYD 1582, HYD 1584, ST 708, ST 722, ST 342, ST 349, HYD 1625, and HYD 1666, preferably MO 245, HE 800, GG1, HYD 721 and ST 716.

16. The nucleus according to claim 3, characterized in that the EPS are chosen among HE 800, EPS 721, MO245, GG1, HYD 657, HYD 1644, HYD 1545, GY 785, MS 907, ST 716, HYD 721, GY 772, HYD 750, GY 768, GY 788, BI746, GY 786, GY 685, GY 686, ST 719, HYD 1574, HYD 1579, HYD 1582, HYD 1584, ST 708, ST 722, ST 342, ST 349, HYD 1625, and HYD 1666, preferably MO 245, HE 800, GG1, HYD 721 and ST 716.

17. The nucleus according to claim 4, characterized in that the bactericidal or bacteriostatic agent(s) is (are) chosen among the antimicrobial peptides (AMP), preferably from tachyplesin or the oyster defensins Cg-Defs.
